(19) United States
(12) Patent Application Publication
(10) Pub. No.: US 2014/0347631 A1
(43) Pub. Date: Nov. 27, 2014

(54) OPHTHALMOLOGIC IMAGING APPARATUS AND CONTROL METHOD THEREFOR

(71) Applicant: CANON KABUSHIKI KAISHA, Tokyo (JP)

(72) Inventors: Nobuyoshi Kishida, Nishitokyo-shi (JP); Toshiya Fujimori, Kawasaki-shi (JP)

(73) Assignee: CANON KABUSHIKI KAISHA, Tokyo (JP)

(21) Appl. No.: 14/278,097

(22) Filed: May 15, 2014

(30) Foreign Application Priority Data
May 23, 2013 (JP) 2013-109013
May 23, 2013 (JP) 2013-109014

(51) Int. Cl. A61B 3/15; A61B 3/12; A61B 3/14

(52) U.S. Cl. CPC A61B 3/152; A61B 3/14; A61B 3/12
USPC 351/208; 351/206; 351/246

(57) ABSTRACT

Provided is an ophthalmologic imaging apparatus capable of imaging with reduced imaging time based on a combination of an imaging type and a mydriatic state of an eye to be inspected. The ophthalmologic apparatus includes an acquiring unit configured to acquire information about a mydriatic state of a pupil of a person to be inspected, and a determining unit configured to determine an imaging order for imaging one of left and right eyes of the person to be inspected a plurality of times and imaging the other eye in accordance with the information about the mydriatic state acquired by the acquiring unit.

### BACKGROUND OF THE INVENTION

1. Field of the Invention

The present invention relates to an ophthalmologic imaging apparatus such as a fundus camera used in a group examination, an ophthalmological clinic, or the like, and to a control method for an ophthalmologic imaging apparatus.

2. Description of the Related Art

Hitherto, fundus imaging by a fundus camera has been widely used for a purpose of screening in a group examination or a diagnosis of ophthalmological diseases. In recent years, a method of recording fundus images as digital data has been commonly used in general. The imaged data is recorded in a portable recording medium, a hard disk drive of a PC, or the like.

In addition, in some cases, the fundus imaging is performed while a pupil of an eye to be inspected is dilated by using a mydriatic agent or the like, and in other cases, the fundus imaging is performed on a naturally dilated pupil without using a mydriatic agent or the like.

Types of imaging using a fundus camera include general color imaging, autofluorescence imaging (hereinafter referred to as FAF imaging) for imaging fundus autofluorescence, stereo imaging for acquiring a three-dimensional fundus image by utilizing parallax, and anterior imaging for imaging an iris or a pupil.

For instance, in fundus imaging in an ophthalmological clinic, the FAF imaging may be performed in addition to color imaging, so as to make a diagnosis on the eye to be inspected in a multilateral manner.

In this way, there are several types of imaging using a fundus camera. Therefore, a photographer may be confused about which fundus imaging should be performed or may forget to take a fundus image necessary for diagnosis. In addition, there is also a problem in that an imaging procedure becomes complicated.

In order to solve the above-mentioned problem, in Japanese Patent Application Laid-Open No. 2010-5073, there is disclosed a medical imaging apparatus in which an imaging sequence as the imaging procedure is registered in advance and displayed so that a photographer can easily know which fundus imaging should be performed in the imaging operation.

Using the fundus camera disclosed in Japanese Patent Application Laid-Open No. 2010-5073, a photographer can know which fundus imaging should be performed in the imaging operation, but this selection does not always consider imaging time.

For instance, it is supposed that the color imaging and the FAF imaging are performed on both eyes to be inspected without using a mydriatic agent.

In an imaging sequence display screen, there are displayed (No. 1, color imaging, right eye), (No. 2, color imaging, left eye), (No. 3, FAF imaging, right eye), (No. 4, FAF imaging, left eye), and the like. Therefore, it is possible to prevent omission of imaging. However, when imaging is performed in the order of (No. 1, color imaging, right eye), (No. 3, FAF imaging, right eye), (No. 2, color imaging, left eye), and (No. 4, FAF imaging, left eye), miosis occurs between (No. 1, color imaging, right eye) and (No. 3, FAF imaging, right eye), and hence it is necessary to wait until the eye to be inspected is dilated. The same is true between (No. 2, color imaging, left eye) and (No. 4, FAF imaging, left eye), and hence it is also necessary to wait until the eye to be inspected is dilated.

In this case, imaging efficiency may be lowered for the examiner, and imaging time may be prolonged for the person to be inspected.

Further, there is a problem in that it is necessary to manually perform alignment between the eye to be inspected and the fundus camera main body, focus adjustment to the fundus of the eye to be inspected, and the like, which is complicated for the examiner.

### SUMMARY OF THE INVENTION

It is an object of the present invention to provide an ophthalmologic imaging apparatus capable of shortening imaging time and resolving a complicated operation for an examiner.

In order to achieve the above-mentioned object, an ophthalmologic imaging apparatus according to one aspect of the present invention includes: an illumination optical system configured to illuminate an eye to be inspected; an imaging optical system configured to observe and image the eye to be inspected through an objective lens and a focus lens; a focus index disposed in the illumination optical system so as to project light to a fundus of the eye to be inspected; a focus detection portion configured to detect a focus state from the focus index; a focus drive unit configured to drive the focus lens based on the focus detection portion; an alignment detection portion configured to detect a positional state between the eye to be inspected and the apparatus; an alignment drive unit configured to drive an alignment mechanism based on the alignment detection portion; a left and right eye detection unit including a left and right eye detection mechanism for determining whether the eye to be inspected is a left eye or a right eye; and a mydriatic state detection unit configured to detect a mydriatic state of the eye to be inspected. The ophthalmologic imaging apparatus is configured to image one of eyes a plurality of times and image left and right eyes. The mydriatic state detection unit switches an imaging sequence.

According to one embodiment of the present invention, it is possible to shorten the imaging time and resolve the complicated operation for the examiner. Therefore, it is possible to improve imaging efficiency for the examiner, and it is possible to reduce imaging load on the person to be inspected.

Further features of the present invention will become apparent from the following description of exemplary embodiments with reference to the attached drawings.

### BRIEF DESCRIPTION OF THE DRAWINGS

FIG. 1 is a structural diagram of a fundus camera according to a first embodiment of the present invention.

FIG. 2 is a structural diagram of an optical system constructing an imaging portion of the fundus camera.

FIGS. 3A and 3B each illustrate an observed image on a two-dimensional imaging element of an anterior observation optical system.

FIGS. 4A and 4B each illustrate an observed image on the imaging element.

FIGS. 5A and 5B are each an imaging sequence when imaging left and right eyes.

FIG. 6 is a diagram illustrating a pupil portion P and a pupil diameter PL′ thereof after color imaging.

FIGS. 7A and 7B are each an imaging sequence when performing stereo imaging of both eyes.

### DESCRIPTION OF THE EMBODIMENTS

Now, embodiments of the present invention are described in detail with reference to the attached drawings.

### First Embodiment

The present invention is described in detail based on the illustrated embodiment.

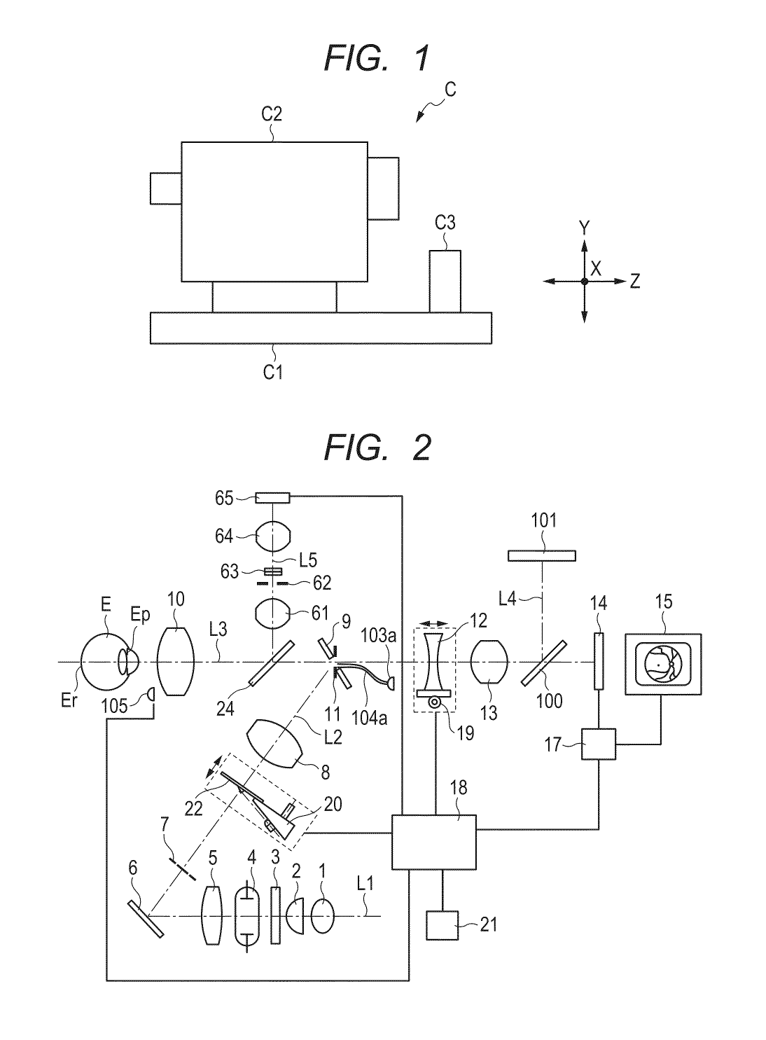

FIG. 1 is a structural diagram illustrating a fundus camera according to this embodiment as an ophthalmologic imaging apparatus. A fundus camera C includes a base unit C1, and an imaging portion C2 including an optical system movable in a left and right direction (X direction), in a front and rear direction (operating distance, Z direction), and in an up and down direction (Y direction) with respect to the base unit C1.

The imaging portion C2 is configured to move in three-dimensional (XYZ) directions with respect to an eye to be inspected E by a drive portion including a pulse motor or the like disposed in the base unit C1. In addition, the imaging portion C2 can be moved in the XYZ directions by operation of a joy stick C3.

Next, an optical system constructing the imaging portion C2 of the fundus camera C is described with reference to FIG. 2. The optical system corresponds to an imaging optical system configured to observe and image the eye to be inspected.

On an optical axis L1, there are disposed an observation light source 1 configured to emit fixed light from a halogen lamp or the like, a condenser lens 2, a filter 3 configured to transmit infrared light and to block visible light, an imaging light source 4 such as a strobe light, a lens 5, and a mirror 6. On an optical axis L2 in the direction of reflection by the mirror 6, there are arranged in order a ring stop 7 having a ring-like aperture, a relay lens 8, and a perforated mirror 9 having a center aperture.

In addition, on an optical axis L3 in the direction of reflection by the perforated mirror 9, there are disposed a dichroic mirror 24 and an objective lens 10 so as to be opposed to the eye to be inspected E. The dichroic mirror 24 can be inserted in or removed from the optical axis L3. Then, an imaging stop 11 is disposed in the aperture of the perforated mirror 9. Further, behind the imaging stop 11, there are arranged in order a focus lens 12 that moves on the optical axis L3 so as to adjust focus, an imaging lens 13, and a half mirror 100. Behind the half mirror 100, there is disposed an imaging element 14 for observing a moving image and for taking a still image. At the end of an optical axis L4 in the direction of reflection by the half mirror 100, there is disposed an internal fixation lamp 101.

Further, an output of the imaging element 14 is connected to an image processing portion 17, and an output of the image processing portion 17 is connected to a system control portion 18. The image processing portion 17 outputs an observed image imaged by the imaging element 14 to be displayed on a monitor 15.

Now, structures of optical systems for alignment and focus are described.

First, a structure of an anterior observation optical system for alignment is described.

On an optical axis L5 in the direction of reflection by the dichroic mirror 24, there is formed an anterior observation optical system for observing an anterior ocular segment, which includes a lens 61, a stop 62, a prism 63, a lens 64, and a two-dimensional imaging element 65 having sensitivity in an infrared region. Here, light entering the prism 63 is refracted and split by an upper half and by a lower half of the prism 63 oppositely in the left and right direction. Therefore, if a distance between the eye to be inspected E and the imaging portion C2 is longer than an appropriate operating distance, a position of an image formed by the lens 61 becomes closer to the lens 61 than the prism 63. Then, an upper half of the observed image is shifted to right while a lower half of the observed image is shifted to left. Using this anterior observation optical system, it is possible to observe the anterior ocular segment of the eye to be inspected E illuminated by an anterior observation light source 105 in an infrared wavelength region different from the wavelength passing through the filter 3 for blocking visible light, so as to detect the alignment state with the anterior ocular segment of the eye to be inspected E.

Next, a structure of an alignment index projection optical system is described.

On a front surface of the perforated mirror 9, there is disposed a light emitting end of a light guide 104a for guiding light beams from an LED light source 103a, and this light emitting end is an alignment index P1. The alignment index P1 is disposed away from the optical axis L3. In addition, at a symmetric position with respect to the alignment index P1 around the optical axis L3, there is disposed a light emitting end of a light guide 104b for guiding light beams from an LED light source 103b (not shown) having the same wavelength as the LED light source 103a. This light emitting end is an alignment index P2 constructing the alignment index projection optical system. Further, if the operating distance between the eye to be inspected E and the imaging portion C2 is appropriate, light beams from the light emitting ends of the light guide 104a and the light guide 104b are reflected by a corneal surface of the eye to be inspected E. Then, the index light beams become parallel and propagate the same optical path as a fundus reflection light beam of an illumination light beam, so as to form images on an image plane of the imaging element 14. Using the alignment index projection optical system described above, it is possible to detect a positional relationship between the alignment indexes so that an alignment state with a fundus Er of the eye to be inspected E can be detected.

Finally, a structure of a focus optical system is described.

Between the ring stop 7 and the relay lens 8 on the optical axis L2, there is disposed a focus index projecting portion 22. This focus index projecting portion 22 is disposed for projecting a split index on a pupil Ep of the eye to be inspected E. Further, the focus index projecting portion 22 and the focus lens 12 are configured to synchronously move in directions of the optical axis L2 and the optical axis L3 respectively by a focus lens drive portion 19 and a focus index drive portion 20 based on control by the system control portion 18. In this case, the focus index projecting portion 22 and the imaging element 14 have an optically conjugate relationship. Using this focus optical system, it is possible to detect a focus state of the fundus Er of the eye to be inspected E.

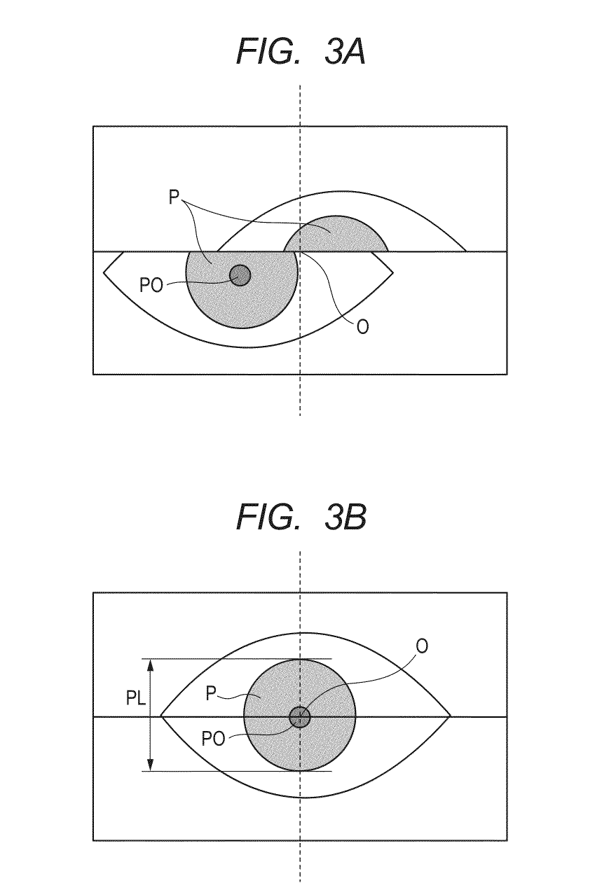

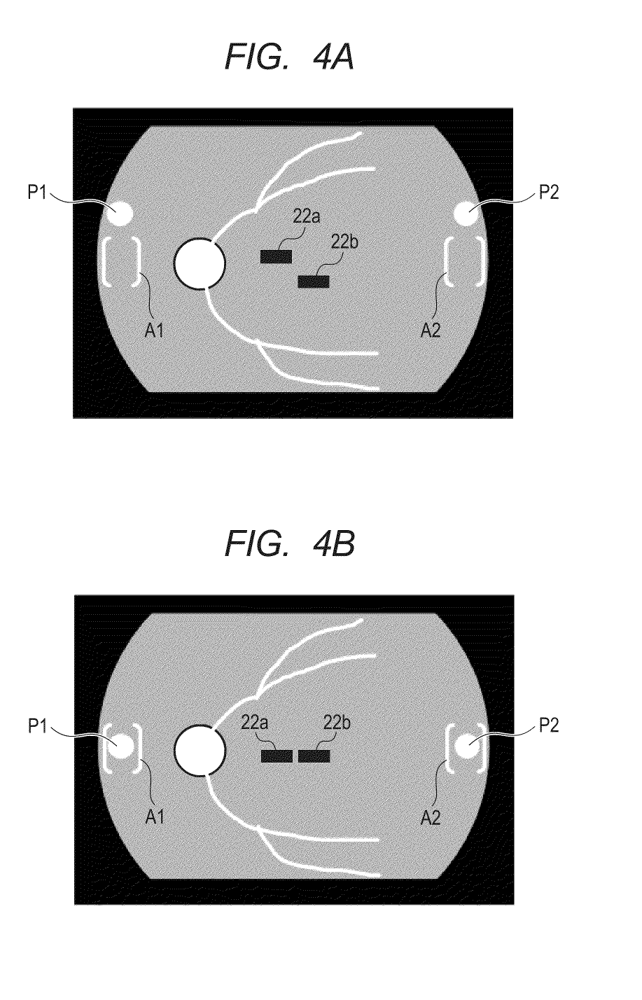

The structures of the alignment optical system and the focus optical system are described above. Next, operations thereof are described in more detail with reference to FIGS. 3A, 3B, 4A and 4B.

FIGS. 3A and 3B illustrate observed images on the two-dimensional imaging element 65 of the anterior observation optical system illustrated in FIG. 2. The anterior ocular segment of the eye to be inspected E illuminated by the anterior observation light source 105 is split by the prism 63 into upper and lower parts and is observed on the two-dimensional imaging element 65 as illustrated in FIG. 3A. A part other than the pupil is displayed in white because much reflection light of the anterior observation light source 105 enters, while the pupil is displayed in black because reflection light does not enter. Therefore, this contrast difference enables extraction of a pupil portion P so that a pupil position is determined. In FIG. 3A, a pupil center PO is detected from a lower part of the pupil portion P split into upper and lower parts. The drive portion disposed in the base unit C1 is operated so that the pupil center PO detected in this way is located in an image center O of the two-dimensional imaging element 65 illustrated in FIG. 3B, and hence alignment of the anterior ocular segment of the eye to be inspected E can be automatically performed.

Next, FIGS. 4A and 4B illustrate the observed images on the imaging element 14, which is used both for observing a moving image and for taking a still image as described above with reference to FIG. 2. The alignment indexes P1 and P2 are bright spots of the LED light source 103a and the LED light source 103b described above for the alignment index projection optical system. A guide frame A1 and a guide frame A2 indicate alignment positions of the alignment indexes P1 and P2, respectively. In addition, split indexes 22a and 22b indicate indexes split on the pupil of the eye to be inspected E and projected by the focus index projecting portion 22 of the focus optical system.

When an automatic alignment is performed on the anterior ocular segment of the eye to be inspected E so as to achieve the state of FIG. 3B, the alignment indexes P1 and P2 appear near the guide frame A1 and the guide frame A2 resulting in the observation image illustrated in FIG. 4A. Here, the alignment indexes P1 and P2 have higher brightness than the reflection light from the fundus Er of the eye to be inspected E illuminated by the observation light source 1, and therefore can be easily detected by image processing such as binarization of the observed image on the imaging element 14. Then, by operating the drive portion disposed in the base unit C1 so that the alignment indexes P1 and P2 respectively appear within the guide frame A1 and the guide frame A2 as illustrated in FIG. 4B, alignment with the fundus Er of the eye to be inspected E can be automatically performed. The structure for alignment between the eye to be inspected E and the imaging optical system described above constructs an alignment unit.

In addition, the focus index projecting portion 22 and the focus lens 12 synchronously move in the directions of the optical axis L2 and the optical axis L3, respectively, based on control by the system control portion 18. Thus, the imaging element 14 and the focus index projecting portion 22 have an optically conjugate relationship. Therefore, when the focus index projecting portion 22 is moved in the direction of the optical axis L2, the split indexes 22a and 22b are moved in the observed image on the imaging element 14, and the focus lens 12 is synchronously moved in the direction of the optical axis L3. In other words, by controlling the split indexes 22a and 22b to move on the imaging element 14 from the state of FIG. 4A to the state of FIG. 4B (in a straight line), focus on the fundus Er of the eye to be inspected E can be automatically performed. The structure for obtaining the focus state of the imaging optical system with respect to the eye to be inspected as described above constructs a focus unit.

Further, in the present invention, it is necessary to determine in advance whether the eye to be inspected as a subject to imaging and observation is the right eye or the left eye as described later. As a determination method, for example, it is possible to determine based on an image of the eye to be inspected under being observed, or an operator may input information of the eye from which the inspection should be started in advance so that the determination is made based on the input. The structure described above constructs a left and right eye detection unit for determining whether the eye to be inspected is the left or right eye in the present invention.

As described above, the fundus camera according to this embodiment can automatically perform all the operations from alignment through focusing by operations of the alignment and the focusing. In addition, it should be understood that imaging operation can be performed after detecting that the alignment operation and the focusing operation are finished. In other words, the examiner can perform automatic imaging using the fundus camera C by pressing an imaging start switch (not shown) in an operation input portion 21. Further, because the fundus camera C can move in the left and right direction (X direction), it is also possible to automatically switch the eye to be imaged. With this structure, this embodiment is described as an ophthalmologic imaging apparatus, which can automatically perform the alignment operation and the focus operation to the eye to be imaged so as to automatically perform the operation until imaging.

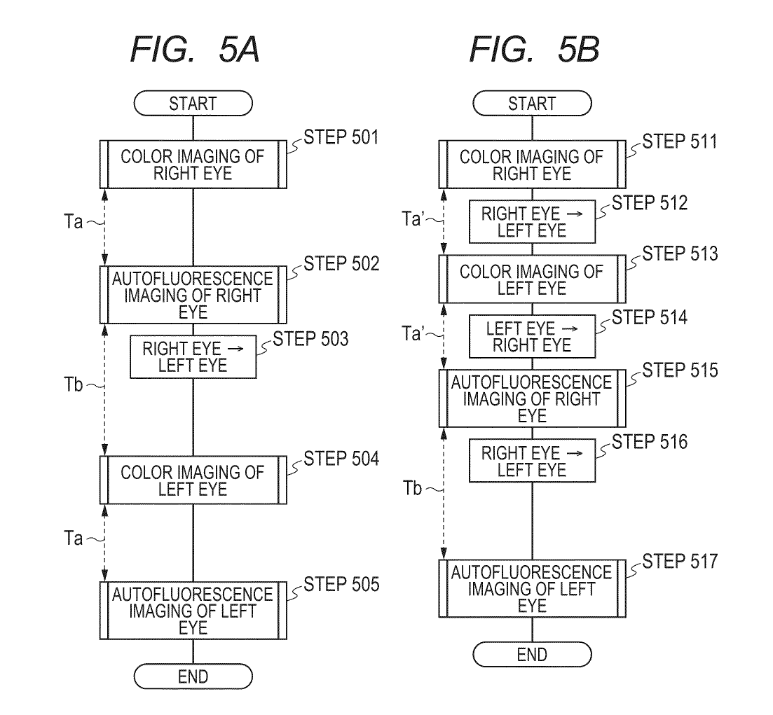

Next, a characteristic operation in this embodiment is described with reference to FIGS. 5A and 5B.

FIGS. 5A and 5B illustrate imaging sequences when imaging of the same eye to be inspected is performed in two imaging modes of the color imaging and the autofluorescence imaging for the left and right eyes. Here, the imaging sequence means an imaging procedure including at least one of an imaging type, information about whether or not imaging of the left and right eyes is performed, and information about whether or not a mydriatic agent is used. Here, there is described a case where the imaging is performed without a mydriatic agent to the left and right eyes in order to reduce load on the person to be inspected.

Hitherto, as illustrated in the imaging sequence of FIG. 5A, color imaging of the right eye is performed in Step 501, and autofluorescence imaging of the right eye is performed in Step 502. After that, the eye to be imaged is changed to the left eye in Step 503. Then, color imaging of the left eye is performed in Step 504, and autofluorescence imaging of the left eye is performed in Step 505. The right eye is first imaged in the imaging sequence described above, but it should be understood that there is a case where the left eye is first imaged.

In this way, hitherto, after finishing imaging two times for one eye (the right eye in FIG. 5A), imaging is performed two times for the other eye (the left eye in FIG. 5A). However, when the imaging is performed without a mydriatic agent, miosis of the pupil of the person to be inspected occurs after color imaging of the right eye in Step 501. In other words, after finishing the color imaging of the right eye in Step 501, it is necessary to wait until the pupil of the person to be inspected is dilated for time Ta before starting the autofluorescence imaging of the right eye in Step 502. Similarly, after finishing the color imaging of the left eye in Step 504, it is necessary to wait until the pupil of the person to be inspected is dilated for the time Ta before starting the autofluorescence imaging of the left eye in Step 505.

In addition, after finishing the autofluorescence imaging of the right eye in Step 502, it is necessary to wait until the pupil of the person to be inspected is dilated for time Tb before starting the color imaging of the left eye in Step 504. During this waiting time, switching from the right eye to the left eye is performed in Step 503. Hitherto, because of simplification of alignment operation by the operator, the imaging sequence of FIG. 5A is usually adopted.

On the other hand, as illustrated in the imaging sequence of FIG. 5B, alternate imaging of the left and right eyes is the characteristic operation in this embodiment. The color imaging of the right eye is performed first in Step 511, and switching from the right eye to the left eye is performed in Step 512. When time Ta′ elapses after finishing the color imaging of the right eye in Step 511, the color imaging of the left eye is performed in Step 513. Next, when the time Ta′ elapses after finishing the color imaging of the left eye in Step 513, switching from the left eye to the right eye is performed in Step 514, and the autoflouorescence imaging of the right eye is performed in Step 515. Finally, after finishing the autofluorescence imaging of the right eye in Step 515, switching from the right eye to the left eye is performed in Step 516, and the autofluorescence imaging of the left eye is performed in Step 517.

That is, in this case, as an imaging sequence for shortening imaging time, there are employed (No. 1, color imaging, right eye), (No. 2, color imaging, left eye), (No. 3, FAF imaging, right eye), and (No. 4, FAF imaging, left eye).

After imaging in (No. 1, color imaging, right eye), imaging in (No. 2, color imaging, left eye) with little influence of miosis is performed. The miosis of the right eye due to the last imaging in (No. 1, color imaging, right eye) is ameliorated while the imaging in (No. 2, color imaging, left eye) is performed. Therefore, imaging in (No. 3, FAF imaging, right eye) is performed at a timing capable of imaging in the mydriatic state. Similarly, the miosis of the left eye due to the last imaging in (No. 2, color imaging, left eye) is ameliorated while the imaging in (No. 3, FAF imaging, right eye) is performed. Therefore, imaging in (No. 4, FAF imaging, left eye) is performed at a timing capable of imaging in the mydriatic state.

In this way, the imaging sequence of FIG. 5B is different from the imaging sequence of FIG. 5A in that two steps of the switching from the left eye to the right eye in Step 514 and the switching from the right eye to the left eye in Step 516 are added. Further, the order of the color imaging of the left eye in Step 513 and the autofluorescence imaging of the right eye in Step 515 is opposite. On the other hand, however, it is understood that the time from start to finish of the imaging becomes shorter in the imaging sequence of FIG. 5B than in the imaging sequence of FIG. 5A.

The reason why the time of the imaging sequence can be shortened is described again in detail.

There is utilized the fact that the time until the pupil of the person to be inspected is dilated after imaging is shorter for the eye that is not imaged than for the eye that is imaged. In FIGS. 5A and 5B, the time Ta′ until the pupil of the eye that is not imaged after the color imaging illustrated in FIG. 5B is dilated is shorter than the time Ta until the pupil of the eye that is imaged after the color imaging illustrated in FIG. 5A is dilated. In other words, instead of waiting for the time Ta until the pupil of the right eye is dilated after performing the color imaging of the right eye in Step 511 as illustrated in the imaging sequence of FIG. 5B, the color imaging of the left eye is performed in Step 513 when the time Ta′ elapses until the pupil of the left eye is dilated. Thus, the imaging time can be shortened by the time obtained by subtracting the time Ta′ from the time Ta (time Ta−time Ta′). Similarly, after performing the color imaging of the left eye in Step 513, when the time Ta′ elapses until the pupil of the right eye is dilated, the autofluorescence imaging of the right eye is performed in Step 515. Thus, the imaging time can be shortened by the time obtained by subtracting the time Ta′ from the time Ta (time Ta−time Ta′).

Further, utilizing the time Ta′ until the pupil of the eye that is not imaged is dilated after the color imaging, the switching from the right eye to the left eye in Step 512 and the switching from the left eye to the right eye in Step 514 are performed.

As described above, because the time after color imaging of both eyes until the next imaging can be shortened, the time from start to finish of the imaging becomes shorter in the imaging sequence of FIG. 5B than in the imaging sequence of FIG. 5A. Here, the right eye is first imaged in the imaging sequence illustrated in FIGS. 5A and 5B, but it should be understood that there is a case where the left eye is first imaged.

In addition, in the imaging sequence of FIG. 5B, the two steps of the switching from the left eye to the right eye in Step 514 and the switching from the right eye to the left eye in Step 516 are added to the imaging sequence of FIG. 5A. Therefore, it is likely that the operation becomes complicated. However, as described above, the ophthalmologic apparatus according to this embodiment automatically performs the alignment and focus to the eye to be imaged so as to be capable of automatically performing the operation until imaging. Therefore, the operator is not required to switch the left and right eyes to be imaged or to perform the alignment and focus operations. Thus, complication of the operation is resolved.

As described above, the ophthalmologic imaging apparatus according to this embodiment can reduce load on the person to be inspected because the imaging time can be shortened without requiring the operator to perform complicated operation.

Two imaging modes of the color imaging and the autofluorescence imaging are described in this embodiment, but also in three times of imaging for one eye in which a red-free imaging is combined to the above-mentioned two imaging modes, it is possible to shorten the time from start to finish of the imaging by the imaging sequence in which the other eye is imaged after imaging one eye.

As described above, when the imaging is performed without a mydriatic agent, miosis of the pupil of the person to be inspected occurs after color imaging of the right eye performed in Step 501, for example. However, when imaging is performed with a mydriatic agent, the time from start to finish of the imaging becomes shorter in the imaging sequence of FIG. 5A than in the imaging sequence of FIG. 5B.

This is because that miosis of the pupil of the person to be inspected does not occur even after the color imaging of the right eye is performed in Step 501. Therefore, it is not necessary to secure the time Ta after performing the color imaging of the right eye in Step 501 until the pupil is dilated. Therefore, it is possible to promptly perform the autofluorescence imaging of the right eye in Step 502. Similarly, it is possible to promptly perform the autofluorescence imaging of the left eye in Step 505 after performing the color imaging of the left eye in Step 504.

In other words, because the time Ta and the time Tb until the pupil is dilated are not necessary in the imaging with a mydriatic agent, the time from start to finish of the imaging in the imaging sequence of FIG. 5A is significantly shortened. Therefore, when the imaging sequence of FIG. 5B is performed, the imaging time becomes longer than that in the imaging sequence of FIG. 5A because of the time necessary for the two steps of the switching from the left eye to the right eye in Step 514 and the switching from the right eye to the left eye in Step 516.

Next, a method of solving the above-mentioned problem in the imaging with a mydriatic agent is described.

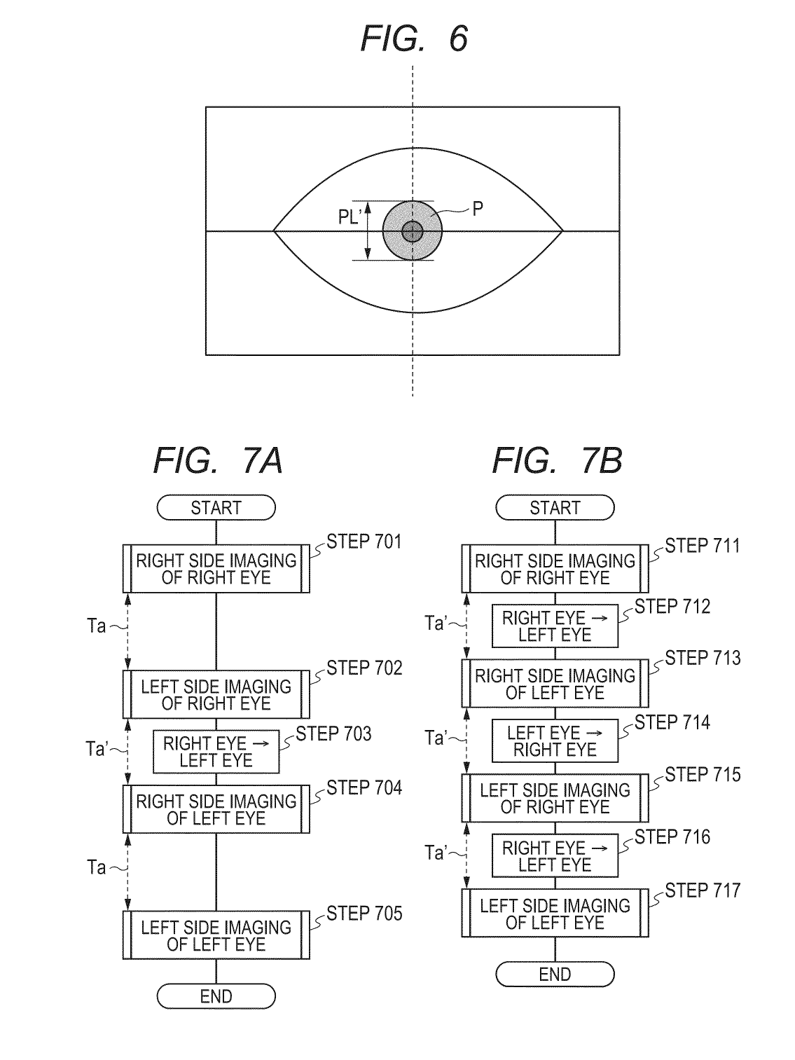

After the color imaging of the right eye of FIG. 5A and FIG. 5B, a size of the pupil of the eye to be inspected is detected. FIG. 6 illustrates the observed image on the two-dimensional imaging element 65 of the anterior observation optical system similarly to FIG. 3B, and indicates the pupil portion P and a pupil diameter PL′ thereof after the color imaging of the right eye. In the observed image on the two-dimensional imaging element 65 before the color imaging of the right eye, the pupil diameter PL′ in FIG. 6 is smaller than the pupil diameter PL in FIG. 3B (pupil diameter PL′<pupil diameter PL). This means that miosis has occurred due to the color imaging of the right eye because a mydriatic agent is not used. In other words, it is possible to detect whether or not a mydriatic agent is used based on the pupil diameter PL′ after the color imaging of the right eye.

Next, an operation by the method described above is described.

When the photography start switch is pressed, the imaging sequence of FIG. 5B is first performed. After the color imaging of the right eye in Step 511 is finished, if the pupil diameter PL′ is smaller than the pupil diameter PL as illustrated in FIG. 6 (pupil diameter PL′<pupil diameter PL), the imaging sequence of FIG. 5B is performed as it is. On the other hand, after the color imaging of the right eye in Step 511 is finished, if the pupil diameter PL′ in FIG. 6 is substantially the same as the pupil diameter PL (pupil diameter PL′≈pupil diameter PL), switching to the imaging sequence of FIG. 5A is performed, and the autofluorescence imaging of the right eye in Step 502 is performed. In this state, the imaging sequence of FIG. 5A is performed. Also with this structure, it is possible to realize the ophthalmologic imaging apparatus in which the imaging sequence of FIG. 5A and the imaging sequence of FIG. 5B can be selected. Further, the detection of the mydriatic state of the eye to be inspected by comparing the pupil diameters as described above is performed by a module region in the system control portion 18 working as a mydriatic state detection unit, which takes images and stores a pupil image of the eye to be inspected, calculates a diameter of the stored pupil image, and compares to evaluate the calculated pupil diameter. The mydriatic state detection unit acquires information about the above-mentioned state of the pupil of the eye to be inspected and detects the mydriatic state based on the information, but it is possible to acquire the mydriatic state from information stored in advance. Therefore, the information about a mydriatic state can be acquired not only by the mydriatic state detection unit but also by reading information from a memory or the like storing patient information. The acquisition of the information is performed by a module region in the system control portion 18 working as an acquiring unit.

Further, the above-mentioned detection of the mydriatic state is performed by an imaging sequence performing unit that selects and performs the imaging sequence in accordance with a result of the detection by the mydriatic state detection unit. The imaging sequence performing unit is constructed by a module region in the system control portion 18. In other words, the module region in the system control portion 18 working as a determining unit images one of the left and right eyes to be inspected for a plurality of times and determines an imaging order for imaging the other eye, in accordance with the acquired information about a mydriatic state.

Further, various known comparing units can be used for the imaging sequence performing unit as long as it is possible to perform detection and comparison of the pupil diameter as described above.

In addition, as the method of solving the above-mentioned problem, the following method may be employed.

Specifically, whether or not a mydriatic agent is used is input to the operation input portion 21 in advance. For instance, there is a method in which the operation input portion 21 includes a mydriatic agent check switch or the like. First, when a mydriatic agent is not used, the mydriatic agent check switch is turned off (is not turned on) while the photography start switch is pressed, thereby performing the imaging sequence of FIG. 5B. On the other hand, when a mydriatic agent is used, the mydriatic agent check switch is turned on while the photography start switch is pressed, thereby performing the imaging sequence of FIG. 5A. In this case, the acquiring unit acquires the information about a mydriatic state input by the input unit. With this structure, it is possible to realize the ophthalmologic imaging apparatus in which the imaging sequence of FIG. 5A and the imaging sequence of FIG. 5B can be selected.

Further, it is preferred that the mydriatic agent check switch for checking whether or not a mydriatic agent has been used work as an imaging sequence input unit for selecting and performing the imaging sequence. Further, a form of the imaging sequence input unit is not limited to the above-mentioned check switch but may be constructed by a selection switch and an execution switch, without limiting to the embodiment.

By the method described above, it is possible to perform the imaging sequence in which the time from start to finish of the imaging becomes shortest depending on with or without a mydriatic agent. Therefore, the effect of reducing load on the person to be inspected can be increased.

As described above, the imaging sequence when imaging the left and right eyes to be inspected for a plurality of times by operating the focus unit and the alignment unit is preferably stored in advance in an imaging sequence storing unit disposed, for example, in the system control portion 18. In addition, it is preferred to store a plurality of the imaging sequences such as ones illustrated in FIG. 5A and FIG. 5B.

### Second Embodiment

In the first embodiment, it is described that the time of the imaging sequence can be shortened in the two imaging modes of the color imaging and the autofluorescence imaging, for example.

In a second embodiment of the present invention, there is described a case where stereo imaging of both eyes to be inspected is performed on the same eye to be inspected.

FIGS. 7A and 7B illustrate imaging sequences when performing the stereo imaging of both eyes to be inspected.

As the stereo imaging by the ophthalmologic imaging apparatus, there are known a simultaneous three-dimensional imaging method in which left and right images used for a three-dimensional image are simultaneously acquired in a single imaging, and an imaging method in which left and right images used for a three-dimensional image are acquired by performing normal imaging two times while shifting in the left and right direction. In this embodiment, there is described a case of adopting the method of performing the normal imaging two times while shifting in the left and right direction. In addition, in this embodiment, the imaging performed two times while shifting in the left and right direction is referred to as right side imaging and left side imaging. Further, there is described a case where the imaging is performed without using a mydriatic agent for the left and right eyes in order to reduce load on the person to be inspected.

Hitherto, as illustrated in the imaging sequence of FIG. 7A, the right side imaging of the right eye is performed in Step 701, and the left side imaging of the right eye is performed in Step 702. Then, the eye to be imaged is changed to the left eye in Step 703. Then, the right side imaging of the left eye is performed in Step 704, and the left side imaging of the left eye is performed in Step 705. The right eye is first imaged in the imaging sequence described above, but it should be understood that there is a case where the left eye is first imaged.

In this way, hitherto, after finishing the stereo imaging two times for one eye (the right eye in FIG. 7A), the stereo imaging is performed two times for the other eye (the left eye in FIG. 7A). However, if the imaging is performed without a mydriatic agent, miosis of the pupil of the person to be inspected occurs after the right side imaging of the right eye is performed in Step 701. In other words, it is necessary to wait until the pupil of the person to be inspected is dilated for the time Ta after finishing the right side imaging of the right eye in Step 701 and before starting the left side imaging of the right eye in Step 702. Similarly, after performing the right side imaging of the left eye in Step 704, it is also necessary to wait until the pupil of the person to be inspected is dilated for the time Ta before starting the left side imaging of the left eye in Step 705.

On the other hand, as illustrated in the imaging sequence of FIG. 7B, alternate imaging of the left and right eyes is the characteristic operation according to this embodiment. First, the right side imaging of the right eye is performed in Step 711, and the switching from the right eye to the left eye is performed in Step 712. Further, when the time Ta′ elapses after the right side imaging of the right eye in Step 711, the right side imaging of the left eye is performed in Step 713. Next, when the time Ta′ elapses after finishing the right side imaging of the left eye in Step 713, the switching from the left eye to the right eye is performed in Step 714. Then, the left side imaging of the right eye is performed in Step 715. Finally, after the left side imaging of the right eye is finished in Step 715, the switching from the right eye to the left eye is performed in Step 716, and the left side imaging of the left eye is performed in Step 717.

Thus, when the imaging sequence of FIG. 7B is compared to the imaging sequence of FIG. 7A, the two steps of the switching from the left eye to the right eye in Step 714 and the switching from the right eye to the left eye in Step 716 are added. Further, the order of the right side imaging of the left eye in Step 713 and the left side imaging of the right eye in Step 715 is opposite. On the other hand, however, it is understood that the time from start to finish of the imaging can be shortened more in the imaging sequence of FIG. 7B than in the imaging sequence of FIG. 7A.

The reason why the time of the imaging sequence can be shortened is the same as that in the first embodiment, and hence description thereof is omitted. However, the time that can be shortened is as follows.

First, instead of waiting for the time Ta until the pupil of the right eye is dilated after the right side imaging of the right eye is performed in Step 711 illustrated in the imaging sequence of FIG. 7B, the right side imaging of the left eye is performed in Step 713 when the time Ta′ until the pupil of the left eye is dilated has elapsed. Thus, the imaging time can be shortened by the time obtained by subtracting the time Ta′ from the time Ta (time Ta−time Ta′). Similarly, after the right side imaging of the left eye is performed in Step 713, the left side imaging of the right eye is performed in Step 715 when the time Ta′ until the pupil of the right eye is dilated has elapsed. Thus, the imaging time can be shortened by the time obtained by subtracting the time Ta′ from the time Ta (time Ta−time Ta′).

Further, utilizing the time Ta′ until the pupil of the eye that is not imaged is dilated after each imaging, the switching from the right eye to the left eye in Step 712 and the switching from the left eye to the right eye in Step 714 are performed.

As described above, the time from start to finish of the imaging can be shortened more in the imaging sequence of FIG. 7B than in the imaging sequence of FIG. 7A. Here, the right eye is first imaged in the imaging sequences illustrated in FIGS. 7A and 7B, but it should be understood that there is a case where the left eye is first imaged. In addition, as described as the embodiments, the imaging sequence includes the color imaging of the eye to be inspected, the autofluorescence imaging for imaging autofluorescence of the fundus, the red-free imaging that is useful for imaging nerve fibers, and the stereo imaging of each of the eyes to be inspected for acquiring left and right images used for a three-dimensional image by performing imaging two times, including the left side imaging from the left direction and the right side imaging from the right direction.

In addition, it should be understood that the same effect as in the first embodiment can be obtained by using the method for solving the problem inherent in the use of a mydriatic agent as described above in the first embodiment.

### Other Embodiments

Further, the present invention can also be realized by performing the following processing. That is, the processing involves supplying software (program) for realizing the functions of the above-mentioned embodiments to a system or an apparatus via a network or various storage media and causing a computer (or a CPU, an MPU, or the like) of the system or the apparatus to read and execute the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-109013, filed May 23, 2013, and Japanese Patent Application No. 2013-109014, filed May 23, 2013, which are hereby incorporated by reference herein in their entirety.

What is claimed is:

1. An ophthalmologic apparatus, comprising:
an acquiring unit configured to acquire information about a mydriatic state of a pupil of a person to be inspected; and
a determining unit configured to determine an imaging order for imaging one of left and right eyes of the person to be inspected a plurality of times and imaging another one of the left and right eyes in accordance with the information about the mydriatic state acquired by the acquiring unit.

2. An ophthalmologic apparatus according to claim 1, further comprising a mydriatic state detection unit configured to detect the mydriatic state in accordance with a state of the pupil,
wherein the acquiring unit acquires a result of detection of the mydriatic state detection unit as the information about the mydriatic state.

3. An ophthalmologic apparatus according to claim 2, further comprising:
an imaging optical system configured to observe and image an eye to be inspected;
a focus unit configured to acquire a focus state of the imaging optical system with respect to the eye to be inspected;
an alignment unit configured to perform alignment between the eye to be inspected and the imaging optical system;
a left and right eye detection unit configured to determine whether the eye to be inspected is a left eye or a right eye;
an imaging sequence storing unit configured to store an imaging sequence including the imaging order when imaging the left and right eyes to be inspected a plurality of times each by operating the focus unit and the alignment unit; and
an imaging sequence performing unit configured to select and perform the imaging sequence in accordance with the acquired information about the mydriatic state.

4. An ophthalmologic apparatus according to claim 3, wherein the imaging sequence storing unit stores the imaging sequence corresponding to a plurality of imaging types.

5. An ophthalmologic apparatus according to claim 3, wherein at least one of the plurality of times of imaging comprises color imaging.

6. An ophthalmologic apparatus according to claim 3, wherein at least one of the plurality of times of imaging comprises autofluorescence imaging.

7. An ophthalmologic apparatus according to claim 3, wherein at least one of the plurality of times of imaging comprises red-free imaging.

8. An ophthalmologic apparatus according to claim 3, wherein the plurality of times of imaging comprises stereo imaging in which the imaging is performed two times while shifting in a left and right direction to acquire left and right three-dimensional images.

9. An ophthalmologic apparatus according to claim 2, wherein the mydriatic state detection unit detects the mydriatic state based on pupil diameters obtained from temporally consecutive images in the imaging sequence that is being performed.

10. An ophthalmologic apparatus according to claim 1, further comprising an input unit,
wherein the acquiring unit acquires the information about the mydriatic state input through the input unit.

11. An ophthalmologic apparatus according to claim 10, further comprising:
an imaging optical system configured to observe and image an eye to be inspected;
a focus unit configured to acquire a focus state of the imaging optical system with respect to the eye to be inspected;
an alignment unit configured to perform alignment between the eye to be inspected and the imaging optical system;
a left and right eye detection unit configured to determine whether the eye to be inspected is a left eye or a right eye;
an imaging sequence storing unit configured to store an imaging sequence including the imaging order when imaging the left and right eyes to be inspected a plurality of times each by operating the focus unit and the alignment unit; and
an imaging sequence input unit configured to select and perform the imaging sequence in accordance with the input information about the mydriatic state.

12. An ophthalmologic apparatus according to claim 11, wherein the imaging sequence input unit inputs whether or not a mydriatic agent is used.

13. An ophthalmologic apparatus according to claim 11, wherein the imaging sequence storing unit stores a plurality of imaging sequences.

14. An ophthalmologic apparatus according to claim 13, wherein the plurality of imaging sequences comprise color imaging of the eye to be inspected.

15. An ophthalmologic apparatus according to claim 13, wherein the plurality of imaging sequences comprise autofluorescence imaging of the eye to be inspected.

16. An ophthalmologic apparatus according to claim 13, wherein the plurality of imaging sequences comprise red-free imaging of the eye to be inspected.

17. An ophthalmologic apparatus according to claim 12, wherein:
the imaging sequence storing unit stores a plurality of imaging sequences; and
the plurality of imaging sequences comprise stereo imaging in which the imaging is performed two times from a left direction and a right direction for each of the left and right eyes to be inspected to acquire left and right three-dimensional images.

18. A control method for an ophthalmologic apparatus, comprising:
acquiring information about a mydriatic state of a pupil of a person to be inspected; and
determining an imaging order for imaging one of left and right eyes of the person to be inspected a plurality of times and imaging another one of the left and right eyes in accordance with the information about the mydriatic state acquired in the acquiring.

19. A control method for an ophthalmologic apparatus according to claim 18, wherein the acquiring comprises acquiring a result of detection by a mydriatic state detection unit configured to detect the mydriatic state in accordance with a state of the pupil as the information about the mydriatic state.

20. A non-transitory tangible medium having recorded thereon a program for causing a computer to perform steps of the control method according to claim 18.
